(19) United States
(12) Patent Application Publication
(10) Pub. No.: US 2025/0352517 A1
(43) Pub. Date: Nov. 20, 2025

(54) THERAPEUTIC OR PROPHYLACTIC AGENT FOR COVID19 WHICH COMPRISES SELENONEINE

(71) Applicants: National University Corporation Chiba University, Chiba (JP); KIKKOMAN CORPORATION, Chiba (JP)

(72) Inventors: Noriyuki Suzuki, Chiba (JP); Yasumitsu Ogra, Chiba (JP); Yasunori Fukumoto, Chiba (JP); Keiichi Ichikawa, Chiba (JP)

(21) Appl. No.: 18/278,142

(22) PCT Filed: Feb. 22, 2022
(86) PCT No.: PCT/JP2022/007397

§ 371 Date: Apr. 2, 2024

(30) Foreign Application Priority Data
Feb. 22, 2021 (JP) 2021-026585
Jun. 25, 2021 (JP) 2021-106076
Oct. 7, 2021 (JP) 2021-165803

(51) Int. Cl. A61K 31/4172; A61P 31/14; C12Q 1/37

(52) U.S. Cl. CPC A61K 31/4172; A61P 31/14; C12Q 1/37

(57) ABSTRACT

An object is to provide a composition for treatment or prevention of an infection caused by SARS-COV-2 (COVID-19). Based on establishment of a screening method for drugs having an inhibitory activity against proteases of SARS-COV2, and discovery of an inhibitory activity of selenoneine against the proteases of SARS-COV2, a composition for treatment or prevention of COVID-19, which composition includes selenoneine, is provided.

### TECHNICAL FIELD

The present invention relates to a technical field of treatment or prevention of COVID-19, including selenoneine.

### BACKGROUND ART

COVID-19 is caused by the SARS-COV-2 virus, which has caused a pandemic from autumn 2019 to 2022. Development of vaccines and therapeutic agents for COVID-19 is urgently needed. Several vaccines have been found to be effective, and their development has proceeded to enable practical use of the vaccines. On the other hand, it has been epidemiologically shown that COVID-19 patients in Asian countries including Japan show lower severity rates and mortality rates. It has thus been thought that there may be a certain factor that imparts resistance to COVID-19. Studies have been carried out on candidates of such a factor. It is known that selenium deficiency in the body leads to increased toxicity of RNA viruses such as Coxsackie virus and influenza virus. Because of such a background, association of the selenium state with the outcome of COVID-19 patients among cities in China has been reported (NPL 1: Am J Clin Nutr. 2020 Jun. 1;111 (6): 1297-1299. doi: 10.1093/ajcn/nqaa095).

Coronavirus has single-stranded RNA as the genome, and infection of a host cell with the virus results in translation of a long polyprotein from the RNA genome. By appropriate cleavage of the polyprotein, each resulting fragment can function as a structural protein or an enzyme required for the viral growth, and this allows the virus to grow. The cleavage of the polyprotein is mainly catalyzed by proteases including the main protease (M^{pro}) and papain-like protease (PLpro). These proteases can be promising potential drug targets. As a result of computer screening of three-dimensional models by crystal analysis, several existing drugs have been reported to be capable of functioning as effective M^{pro} inhibitors (NPL 2: Nature (2020) vol. 582(7811): 289-293). In particular, ebselen, which is a selenium compound, shows a remarkable affinity to the catalytic region, and development of ebselen as a therapeutic agent for COVID-19 has therefore been expected (NPL 3: Sci. Adv. 2020 6. eadb0345). Ebselen is a functional molecule that contains selenium, which is an essential trace element, in the molecule. Ebselen is a molecule that potentially contains a free selenol group as a tautomer. The mechanism of the M^{pro} inhibition by ebselen has been thought to be based on covalent bonding of the selenol group of ebselen to the thiol group at Cys145, which is located at the active center of the protease. Further, organoselenium compounds have been shown to be potential candidate molecules of antiviral drugs since organoselenium compounds show high binding affinities to the main protease (M^{pro}) of SARS-COV-2 in in silico analysis (NPL 4).

### CITATION LIST

### Non Patent Literature

[NPL 1] Am J Clin Nutr. 2020 Jun. 1;111 (6): 1297-1299. doi: 10.1093/ajcn/nqaa095

### [NPL 2] Nature (2020) vol. 582 (7811): 289-293

[NPL 3] Sci. Adv. 2020 6. eadb0345
[NPL 4] Chemrexiv (2020-07-02) DOI: 10.26434/chemrxiv.12594134

### SUMMARY

### Technical Problem

An object is to provide a drug having an inhibitory activity against a protease of SARS-CoV-2, which protease is a drug target.

### Solution to Problem

The present inventors focused attention on the fact that both the main protease (M^{pro}) and papain-like protease (PLpro) of SARS-COV-2 are cysteine proteases, and established a screening system using a papain inhibitory activity as an index. By such screening, selenoneine was identified as a substance having a higher inhibitory activity than ebselen, which had been identified as an inhibitor of M^{pro}. Further, the present inventors prepared M^{pro} of SARS-COV-2, and confirmed that its protease activity is inhibited by selenoneine, thereby reaching the present invention. In view of this, the present invention relates to the following.

[1] An inhibiting agent for protease of coronavirus, comprising selenoneine, a tautomer or dimer thereof, or a pharmaceutically acceptable salt thereof.
[2] The inhibiting agent for protease according to item 1, wherein the protease is the main protease or papain-like protease.
[3] The inhibiting agent for protease according to item 1, wherein the coronavirus is SARS-CoV2.
[4-1] A composition for treatment or prevention of coronavirus infection, comprising selenoneine, a tautomer or dimer thereof, or a pharmaceutically acceptable salt thereof.
[4-2] Selenoneine, a tautomer or dimer thereof, or a pharmaceutically acceptable salt thereof, for use in treatment or prevention of coronavirus infection.
[4-3] A method of treatment or prevention of coronavirus infection in a subject in need of the treatment or prevention of infection with coronavirus, the method comprising administering selenoneine, a tautomer or dimer thereof, or a pharmaceutically acceptable salt thereof to the subject.
[4-4] Use of selenoneine, a tautomer or dimer thereof, or a pharmaceutically acceptable salt thereof, for the production of a therapeutic agent or prophylactic agent for coronavirus.
[5] The invention according to any one of items [4-1] to [4-4], wherein the coronavirus infection is COVID-19.
[6] A method of screening a therapeutic or prophylactic agent for coronavirus infection, using an inhibitory activity against papain as an index.
[7] The method according to item 6, wherein the coronavirus infection is COVID-19.
[8] The method according to item 6 or 7, wherein the therapeutic agent or prophylactic agent inhibits the main protease or papain-like protease of coronavirus.

### Advantageous Effects of Invention

Selenoneine exhibits a higher M^{pro} inhibitory activity than ebselen. Further, selenoneine exhibits a higher M^{pro} inhibitory activity than other selenium-containing compounds.

### BRIEF DESCRIPTION OF THE DRAWINGS

FIG. 1 is a diagram showing a three-dimensional model which illustrates that ebselen acts on the active center of M^{pro}. A selenol group that appears by tautomerization of ebselen covalently binds to cysteine 145.

FIG. 2 shows a comparison between (1) the sequence, and the spatial structure of the active center, of the cysteine protease M^{pro} and (2) the sequence, and the spatial structure of the active center, of the cysteine protease papain.

FIG. 3 shows a comparison between (1) the sequence, and the spatial structure of the active center, of the cysteine protease PLpro and (2) the sequence, and the spatial structure of the active center, of the cysteine protease papain.

FIG. 4 shows papain enzyme activity in the presence or absence of ebselen or selenoneine. Due to the papain enzyme activity, the fluorescence intensity increases over time due to degradation of a fluorescent substrate.

FIG. 5 shows inhibition curves of ebselen and selenoneine against the papain enzyme activity.

FIG. 6 shows a schematic diagram of a plasmid carrying the M^{pro} gene.

FIG. 7 shows a result obtained by subjecting M^{pro} that was expressed by Escherichia coliand purified using a His tag, to SDS-PAGE. The 33.8-kDa band corresponds to the M^{pro} band.

FIG. 8 shows the M^{pro} protease activity in the presence or absence of ebselen, ergothioneine, or selenoneine. The fluorescence intensity increases over time due to degradation of a fluorescent substrate by M^{pro}.

FIG. 9 shows inhibition curves of ebselen, ergothioneine, and selenoneine against the M^{pro} protease activity.

FIG. 10 shows inhibitory activities of test compounds (selenoneine, ebselen, selenocystine ((SeCys)₂), methylselenocysteine (MeSeCys), selenomethionine (SeMet), diphenyl diselenide (PhSeSePh), and sodium selenite (selenite)), against M^{pro}.

### DESCRIPTION OF EMBODIMENTS

Embodiments of the present invention (hereinafter referred to as “present embodiments”) are described below in detail. However, the present invention is not limited to these, and various modifications can be made without departing from the spirit of the present invention.

The present invention relates to an inhibiting agent for protease of coronavirus, or a composition for treatment or prevention of coronavirus infection, the inhibiting agent or composition comprising selenoneine, a tautomer or dimer thereof, or a pharmaceutically acceptable salt thereof.

In the present invention, selenoneine is the following compound having the chemical name 2-selenyl-N_{α},N_{α},N_{α}-trimethyl-L-histidine (2-selenyl-N_{α},N_{α},N_{α}-trimethyl-L-histidine). More specifically, selenoneine is directed to a compound represented by the following Formula (I).

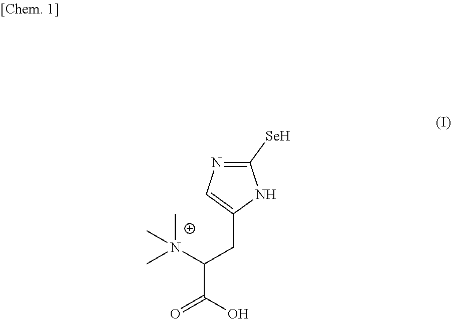

The OH group, NH group, and the like in the molecule may be in a state without the hydrogen atom, that is, in an ionized state. This compound may be present as an arbitrary optical isomer, geometric isomer, tautomer, or dimer, or a mixture thereof. As a result of tautomerization or dimerization, the selenoneine may have any of the forms of the following Formulae (I) to (III).

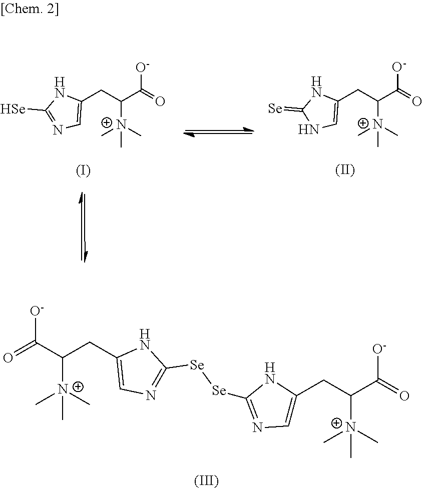

In the present invention, the selenoneine may contain compounds in the forms of Formulae (I) to (III) at arbitrary ratios. The dimerized compound may be reduced to become monomers depending on the surrounding environment. Further, from the viewpoint of maintaining the monomers, a composition comprising a compound represented by Formula (I) or (II) may also comprise a reducing agent. As the reducing agent, an arbitrary reducing agent may be used. Examples of the reducing agent that may be used include glutathione (GSH), dithiothreitol (DTT), and mercaptoethanol. Selenoneine is a component which is abundant in dark meat of fish such as tunas, swordfishes, and mackerels, and which is consumed daily.

Selenoneine can be appropriately produced by those skilled in the art. Selenoneine can be produced by a chemical synthesis method (Angew. Chem. Int. Ed. 2019, 58, 1-6), by extraction from a biological tissue containing selenoneine, or by fermentation by a microorganism. Examples of the production methods include a method described in Japanese Patent Publication No. 5669056 in which selenoneine is extracted from a tissue of a squid, fish, bird, or mammal; a method described in a literature by Pluskal T et al. (Pluskal T et al., PLOS One 2014 May 14;9 (5): e97774) which utilizes a fission yeast Schizosaccharomyces pombe to which a gene involved in an ergothioneine biosynthetic system is introduced; and a method described in WO 2017/026173 in which histidine and a selenium compound are utilized together with a transformant of a microorganism belonging to the genus Aspergillus, for example, Aspergillus sojae, Aspergillus oryzae, or Aspergillus niger, or with a transformant of Escherichia coli or the like, which transformant overexpresses a gene encoding a selenoneine-synthesizing enzyme. In cases where selenoneine is to be produced on an industrial scale, the method described in WO 2017/026173 is preferred from the viewpoint of production of the selenoneine with high yield.

In the method described in WO 2017/026173, which utilizes a transformant that overexpresses a selenoneine-synthesizing enzyme, ergothioneine is produced together with selenoneine. Since these cannot be easily separated from each other, the obtained transformant extract containing selenoneine may contain not only selenoneine, but also ergothioneine. The selenoneine is preferably purified selenoneine in cases where it is available. Selenoneine can be purified by a method known to those skilled in the art, such as HPLC.

The proteases of coronavirus are preferably proteases of SARS-COV2. Examples of the proteases of coronavirus include the main protease (M^{pro}) and papain-like protease (PLpro). The main protease (M^{pro}) of SARS-COV2 is preferred from the viewpoint of the fact that it is a drug target.

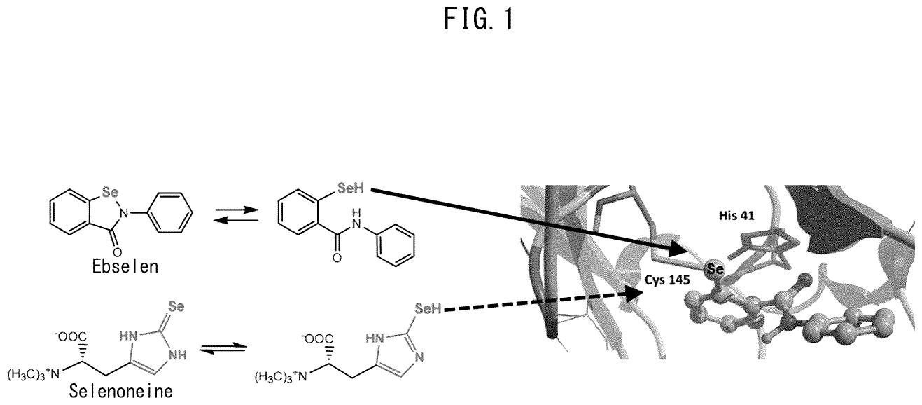

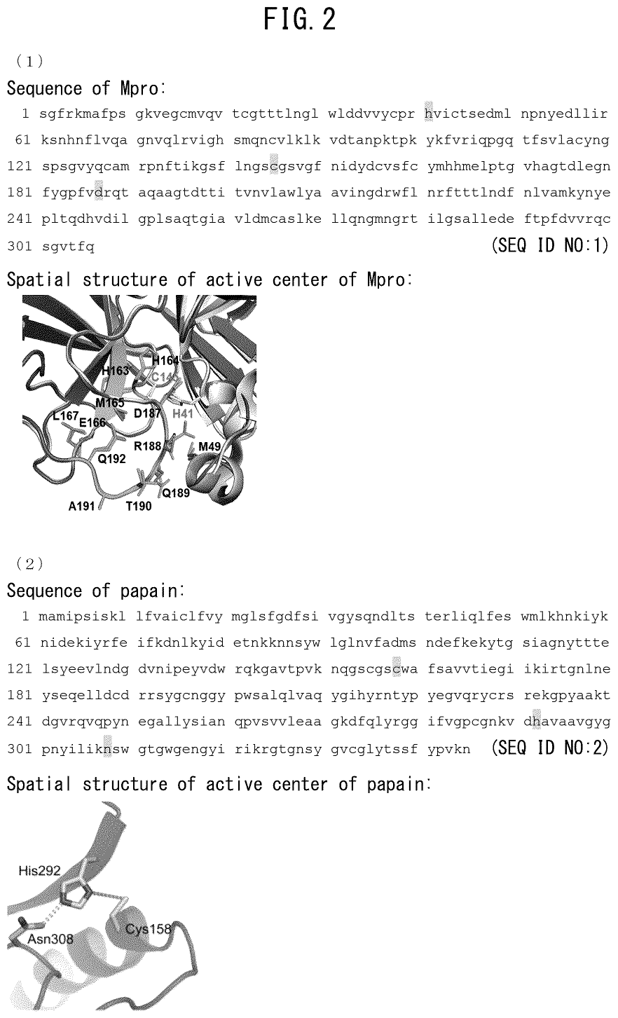

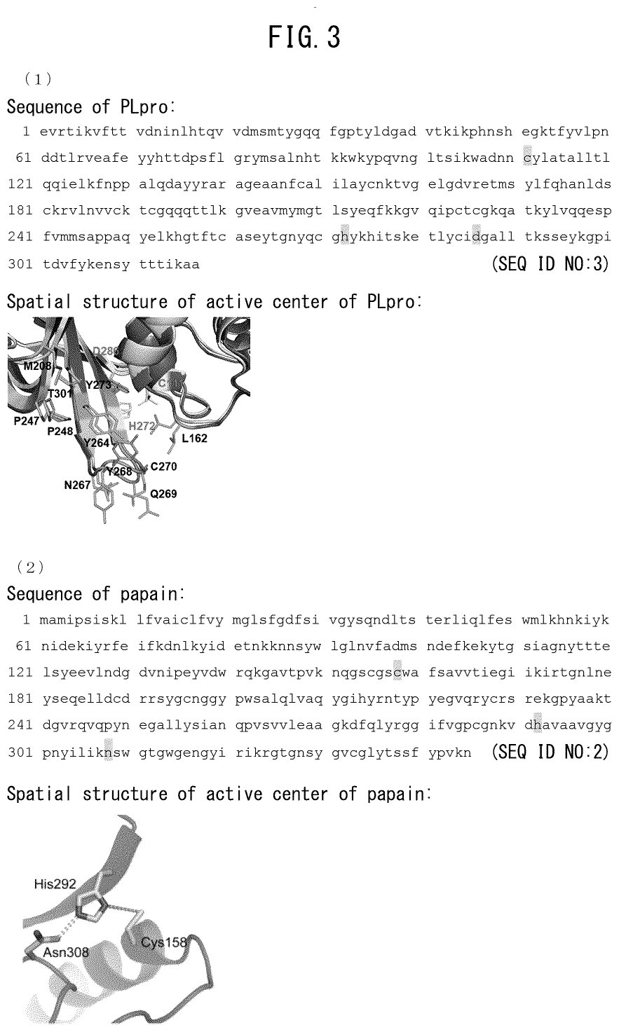

The main protease is also called 3Clpro or nonstructural protein 5 (nsp5), and is a main protease that degrades the polyprotein. The main protease is a cysteine protease, and functions as a dimer composed of the same subunits. The main protease of SARS-COV2 has an amino acid sequence of 306 residues (SEQ ID NO:1). The sequence is known to have an active center formed by Cys145 and His41, and it has been thought that the selenol group of ebselen covalently binds to the thiol group of Cys145 (FIG. 1). Since selenoneine, a compound containing selenium, also has a selenol group, it can covalently bind to Cys145, which forms the active center of M^{pro}. Thus, selenoneine can have an M^{pro} inhibitory activity.

Papain-like protease, which is nonstructural protein 3 (nsp3), is a cysteine protease that degrades the polyprotein. The papain-like protease of SARS-COV2 has an amino acid sequence of 317 residues (SEQ ID NO:3). The catalytic triad structure in the active center of the papain-like protease is Asp286-His272-Cys111.

Papain is a type of cysteine protease contained in papaya. It has an amino acid sequence of 345 residues (SEQ ID NO:2). “Cysteine protease” means a protease containing cysteine in the catalytic region of the enzyme. Usually, deprotonation of the thiol of the cysteine in the catalytic region occurs by histidine present in the vicinity of the cysteine, and the resulting anionic thiol group attacks a carbonyl carbon of a substrate peptide or protein, to hydrolyze a peptide bond. Thus, suppression of the enzymatic activity of a cysteine protease can be achieved by covalent bonding of a protease inhibitor to the thiol group of the cysteine in the catalytic region.

Papain, and the main protease or papain-like protease, are both cysteine proteases, and form a catalytic triad or catalytic dyad characteristically having common amino acid residues in the active center. “Catalytic triad” means three coordinated amino acids found at the active sites of several enzymes. Different types of enzymes have different coordinated amino acids constituting the active site. The catalytic triad of a cysteine protease is formed by cysteine, histidine, and a third amino acid that is asparagine or aspartic acid. Among these, cysteine, and histidine, which contributes to the deprotonation of the thiol of the cysteine, are essential components. However, in cases where the asparagine hardly affects the activity, such as cases of asparagine found in papain and the like, the term “catalytic dyad” is also used since it is formed by the cysteine and the histidine. Therefore, screening for inhibitors of the main protease or papain-like protease is possible by selecting substances having inhibitory activities against papain. Another aspect of the present invention relates to a method of screening for an inhibitor of the main protease or papain-like protease, or for a therapeutic or prophylactic agent for coronavirus infection, using an inhibitory activity against papain as an index. More specifically, the screening method comprises: preparing a solution containing a candidate drug, a papain-degradable fluorescent substrate, and papain; and measuring the fluorescence intensity of the solution. The fluorescence intensity may be measured over time, or changes in fluorescence intensity may be measured while changing the concentration of the candidate drug, to obtain a papain inhibitory curve of the candidate drug. In view of the fact that papain, and the main protease or papain-like protease, exhibit the similarity in the catalytic triad structure or catalytic dyad structure, selenoneine, which shows papain inhibitory activity, may have an inhibitory activity against the main protease or papain-like protease.

The inhibiting agent for protease of the present invention can suppress degradation of the polyprotein produced from coronavirus, to suppress the growth of the coronavirus. Therefore, the inhibiting agent for protease of the present invention can be used as a therapeutic agent or prophylactic agent. Further, the inhibiting agent for protease of the present invention may be contained in a food or food composition.

The composition of the present invention comprises a therapeutically effective amount of selenoneine, a tautomer or dimer thereof, or a pharmaceutically acceptable salt thereof. The composition may also contain a pharmaceutically acceptable carrier or excipient. Therefore, the composition of the present invention can also be said to be a pharmaceutical composition. The inhibiting agent for protease or composition of the present invention is administered to a patient in need of treatment or prevention.

Another aspect of the present invention comprises administering selenoneine, a tautomer or dimer thereof, or a pharmaceutically acceptable salt thereof, or administering an inhibiting agent for protease or a therapeutic or prophylactic composition according to the present invention, to a subject in need of treatment or prevention. The administration may be carried out either orally or parenterally. Examples of the parenteral administration include intraperitoneal administration, intramuscular administration, intravenous administration, intraarterial administration, intranasal administration, buccal administration, pulmonary administration, and topical administration. The dose and the number of doses may be appropriately selected depending on the symptoms.

The term “pharmaceutically acceptable excipient” as used in the present description includes any of carriers, diluents, adjuvants, media, preservatives or antioxidants, fillers, disintegrators, wetting agents, emulsifiers, suspending agents, solvents, dispersion media, coatings, antimicrobial agents, fungicides, isotonic agents, and absorption delaying agents. Use of these excipients for effective components is well-known in the art. Except for cases where conventional excipients cannot coexist with selenoneine, the excipients may be used in the composition of the present invention. Auxiliary effective components may also be incorporated in the composition, to provide an appropriate therapeutic combination.

Examples of the subject in need of treatment or prevention include subjects who may be potentially exposed to coronavirus.

“Therapeutically effective amount” is an amount at which the administration enables inhibition of an active protease involved in development of a disease, and means an amount of a compound/agent according to the present invention at which the administration is effective for prevention or treatment of development or exacerbation of COVID-19. The therapeutically effective amount can be determined through an animal experiment or a clinical trial on humans. On the other hand, selenoneine, the effective component of the present invention, is ingested by eating fish, and, for example, it can be safely used by administration at a daily dose of up to 1.7 mg, so that the therapeutically effective amount may also be determined taking this amount into account. Selenoneine may be administered at, for example, 28 μg/kg, although such a value is not meant to limit the dose.

The composition of the invention may be provided in an arbitrary dosage form, and may be formulated into the form of a tablet, a capsule, a powder, a nasal drop, or an aerosol, or into the form of an injection solution, drops, an ointment, a cream, a spray, or a transdermal patch.

Another aspect of the present invention may relate to a food composition comprising selenoneine, a tautomer or dimer thereof, or a pharmaceutically acceptable salt thereof. Such a food composition may be a food with function claims, a food with nutrient function claims, or a food for specified health uses, with a label indicating a function such as prevention of or resistance to coronavirus, especially SARS-COV2, or a function that inhibits a coronavirus protease, especially the main protease. The food composition, food with function claims, food with nutrient function claims, or food for specified health uses may be, for example, a beverage, food, or supplement that contains selenoneine, a tautomer or dimer thereof, or a pharmaceutically acceptable salt thereof at about 1 to 1000 ppm, preferably at about 10 to 100 ppm, most preferably at about 30 to 70 ppm. Selenoneine is known to be abundant in fish. For example, based on an assumption that the selenoneine content in Pacific bluefin tuna is 30 mg Se/kg, that 100 g of fish meat is ingested, and that selenoneine is uniformly distributed in a human body having a body weight of 60 kg, the selenoneine content in the body can be theoretically assumed to be about 1 μM, and the blood level of selenoneine can be theoretically assumed to be about 8 μM.

Examples of fish that may contain selenoneine include tunas, swordfishes, mackerels, yellowtails, sea breams, puffers, salmons/trouts, and flounders. Selenoneine is especially abundant in tunas, swordfishes, mackerels, and yellowtails. Examples of the food composition, food with function claims, food with nutrient function claims, or food for specified health uses include raw edible parts of these fish, and processed foods using fish as raw materials. These fish may be either wild fish or cultured fish. Since the selenoneine content in fish varies depending on the type of the feed, the fish is more preferably cultured fish having an increased selenoneine content. Examples of the processed foods using fish as raw materials include any foods using fish as raw materials, such as canned foods, bottled foods, tsukudani (fish boiled down in soy sauce), dried fish, dried-fish products (himono), fish pastes (nerimono), pickled fish, and supplements.

Examples of tunas that may be used for the food composition, food with function claims, food with nutrient function claims, or food for specified health uses include the tribe Thunnini and the tribe Sardini. Examples of the tribe Thunnini include the genus Thunnus, the genus Auxis, the genus Euthynnus, and the genus Katsuwonus. Examples of the tribe Sardini include the genus Gymnosarda and the genus Sarda. Examples of the tunas include: albacore, Pacific bluefin tuna, southern bluefin tuna, blackfin tuna, northern bluefin tuna, yellowfin tuna, bigeye tuna, and longtail tuna, which belong to the genus Thunnus; bonito, which belongs to the genus Katsuwonus; frigate tuna and bullet tuna, which belong to the genus Auxis; mackerel tuna, which belongs to the genus Euthynnus; and striped bonito, which belongs to the genus Sarda; or include albacore, Pacific bluefin tuna, southern bluefin tuna, blackfin tuna, northern bluefin tuna, yellowfin tuna, bigeye tuna, longtail tuna, striped bonito, and mackerel tuna. Preferred examples of the tunas include albacore, Pacific bluefin tuna, blackfin tuna, northern bluefin tuna, yellowfin tuna, bigeye tuna, longtail tuna, striped bonito, and mackerel tuna.

All references mentioned in the present description are incorporated herein by reference in their entirety.

The Examples of the present invention described below are merely for illustrative purposes, and do not limit the technical scope of the present invention. The technical scope of the present invention is limited only by the description in Claims. The present invention may be modified by, for example, addition, deletion, and/or substitution of the constituent features of the present invention without departing from the spirit of the present invention.

### EXAMPLES

### Example 1: Inhibitory Activity against Papain Activity

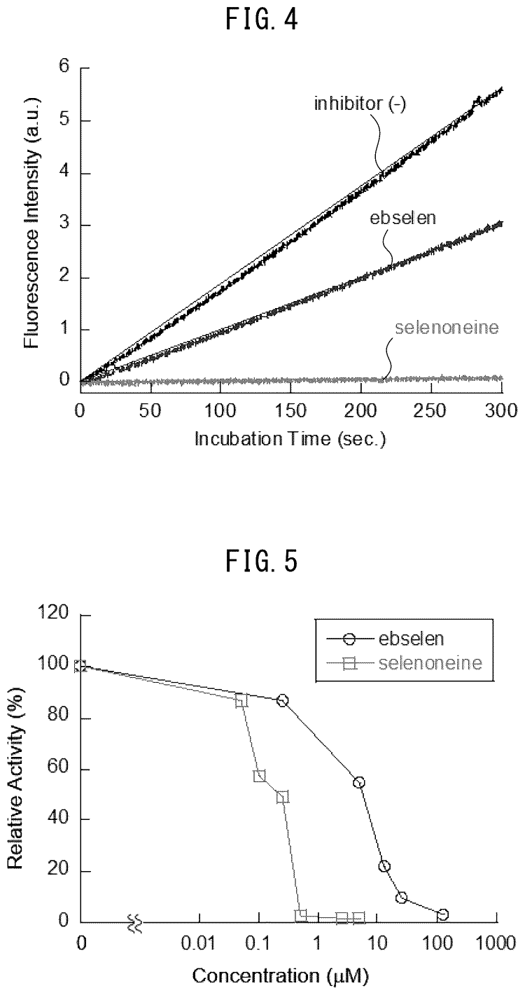

After dissolving 10 μg/ml papain (commercially available from Sigma-Aldrich Japan) and 5 μM ebselen or selenoneine in 50 mM Tris-HCl (pH 7.4) kept at 37° C., preincubation was carried out for 15 minutes. Thereafter, 10 μM Bz-Arg-MCA was added to the reaction solution, and changes in the fluorescence intensity caused by cleavage of Bz-Arg-MCA were observed over time using a fluorometer. The results are shown in FIG. 4.

After dissolving 10 μg/ml papain (commercially available from Sigma-Aldrich Japan) and 0 to 125 μM ebselen or 0 to 5 μM selenoneine in 50 mM Tris-HCl (pH 7.4) kept at 37° C., preincubation was carried out for 15 minutes. Thereafter, 10 μM Bz-Arg-MCA was added to the reaction solution, and changes in the fluorescence intensity caused by cleavage of Bz-Arg-MCA were measured using a fluorometer, to investigate the inhibitory activity. The results are shown in FIG. 5. From the results in FIG. 5, the inhibitory activity of selenoneine (IC₅₀=0.25 μM) and the inhibitory activity of ebselen (IC₅₀=5.0 μM) against papain were determined.

### Example 2: Preparation of M^{pro} of SARS-COV2

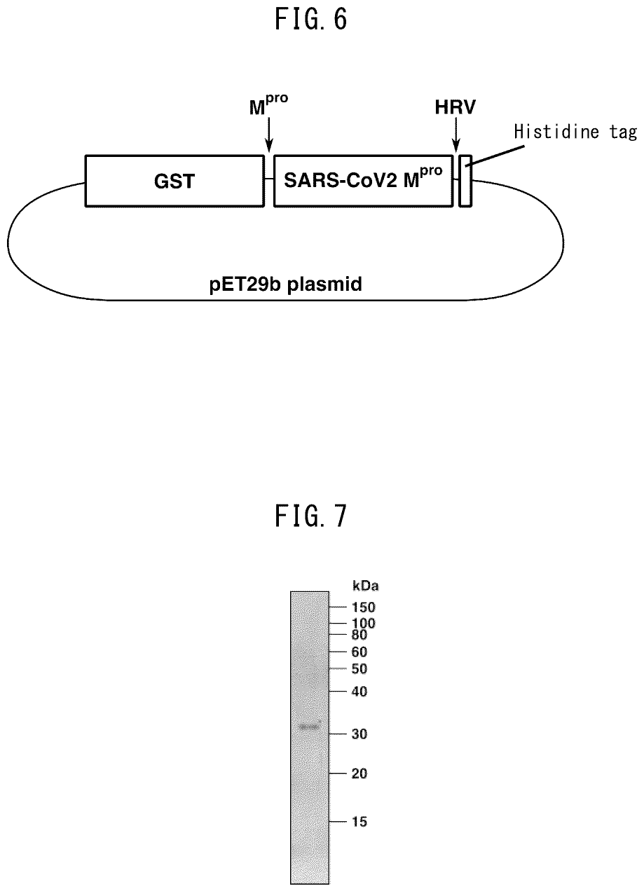

By total gene synthesis, M^{pro} DNA was synthesized according to the base sequence of NC_45512 (10055-10972; SEQ ID NO:4). The M^{pro} was introduced into a plasmid having GST and a histidine tag (FIG. 6), and the resulting plasmid was used for transformation of the E. coli BL21 (DE3) strain. The E. coli BL21 (DE3) strain was cultured at 37° C. to allow expression of M^{pro} protein. The cell bodies were recovered, and M^{pro} protein was purified using a binding agent for the histidine tag. The GST and the histidine tag were removed by autodigestion of M^{pro} and human rhinovirus 3C protease (HRV), and undigested M^{pro} and HRV were removed using a binding agent for the histidine tag, to obtain a purified sample of M^{pro} protein. The purified M^{pro} protein was dissolved in a storage buffer of 20 mM Tris-HCl, 100 mM NaCl, 0.01% Triton-X-100, 50% glycerol, 1 mM EDTA, and 1 mM DTT, and then subjected to SDS-PAGE, followed by staining with Coomassie Brilliant Blue (FIG. 7). The purified M^{pro} protein dissolved in the storage buffer containing DTT was subjected to dialysis using a micro-dialysis cartridge, Xpress Micro/Mini Dialyzer (commercially available from Funakoshi Co., Ltd.), to remove the DTT, followed by subjecting the protein to the inhibitory activity test in Example 4.

### Example 3: Preparation of PLpro of SARS-COV2

By total gene synthesis, PLpro DNA is synthesized according to a base sequence (4955-5908; SEQ ID NO:5) in the sequence accession number NC_45512. The PLpro is introduced into a plasmid having a histidine tag, and the resulting plasmid is used for transformation of the E. coli BL21 (DE3) strain. The E. coli BL21 (DE3) strain is cultured to allow expression of PLpro protein. The cell bodies are recovered, and PLpro protein is purified using a binding agent for the histidine tag. The GST and the histidine tag are removed by autodigestion of PLpro and HRV, and undigested PLpro and HRV are removed using a binding agent for the histidine tag, to obtain a purified sample of PLpro protein.

### Example 4: Inhibitory Activity against M^{pro} of SARS-COV2

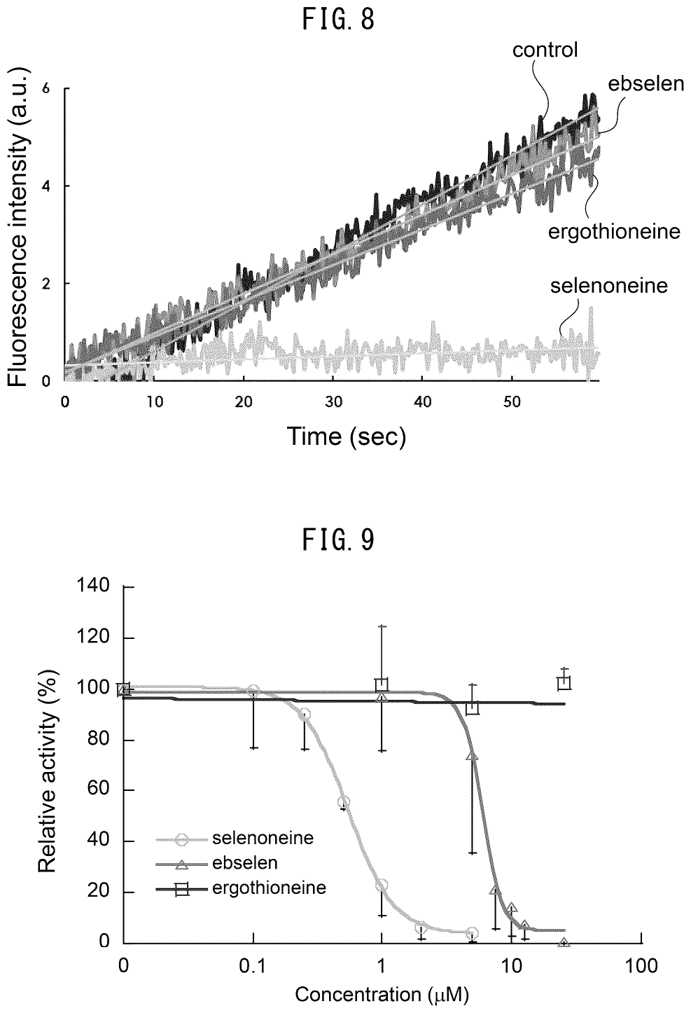

After dissolving 2 μg/ml M^{pro}, and 5 μM ebselen, selenoneine, or ergothioneine, in 50 mM Tris-HCl (pH 7.4) kept at 37° C., preincubation was carried out for 5 minutes. Thereafter, 10 μM Ac-Abu-Tle-Leu-Gln-MCA was added to the reaction solution, and changes in the fluorescence intensity caused by cleavage of Ac-Abu-Tle-Leu-Gln-MCA were measured using a fluorometer, to investigate the inhibitory activity (FIG. 8). In order to provide a control, changes in the fluorescence intensity were measured under the same conditions except that no inhibitory compound was added.

Ac-Abu-Tle-Leu-Gln-MCA is degraded by M^{pro}, to produce a fluorescent substance as follows. The production of the fluorescent substance is suppressed by the action of an M^{pro}-inhibiting compound.

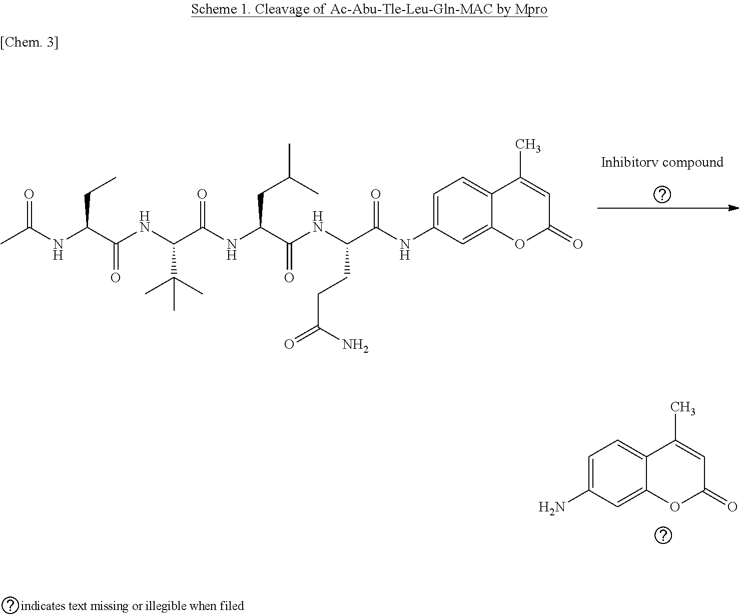 [SMILES: CC[C@H](NC(C)=O)C(=O)N[C@H](C(=O)N[C@@H](CC(C)C)C(=O)N[C@@H](CCC(N)=O)C(=O)Nc1ccc2c(C)cc(=O)oc2c1)C(C)(C)C ; Cc1cc(=O)oc2cc(N)ccc12]

The changes in the fluorescence intensity in the cases of addition of ebselen or ergothioneine were similar to those in the case of the control, in which no inhibitory compound was added. On the other hand, in the case of addition of selenoneine, the fluorescence intensity was lower than that in the case where no inhibitory compound was added, indicating that selenoneine inhibited the activity of M^{pro}.

After dissolving 2 μg/ml M^{pro}, and 0 to 25 μM ebselen, 0 to 25 μM ergothioneine, or 0 to 5 μM selenoneine, in 50 mM Tris-HCl (pH 7.4) kept at 37° C., preincubation was carried out for 5 minutes. Thereafter, 10 μM Ac-Abu-Tle-Leu-Gln-MCA was added to the reaction solution, and changes in the fluorescence intensity caused by cleavage of Ac-Abu-Tle-Leu-Gln-MCA were measured using a fluorometer to measure the fluorescence intensity, to determine an inhibition curve (FIG. 8). The IC₅₀ value was calculated from the regression curve, to obtain the results shown in the following table.

TABLE 1

| IC50 value of each compound calculated | |
|---|---|
| from the regression curve | |
| Compound | IC50 value (μM) |
| Selenoneine | 0.54 |
| Ebselen | 5.92 |
| Ergothioneine | n. d. |

n.d.: not determined

### Example 5: Inhibitory Activity against M^{pro} of SARS-COV2

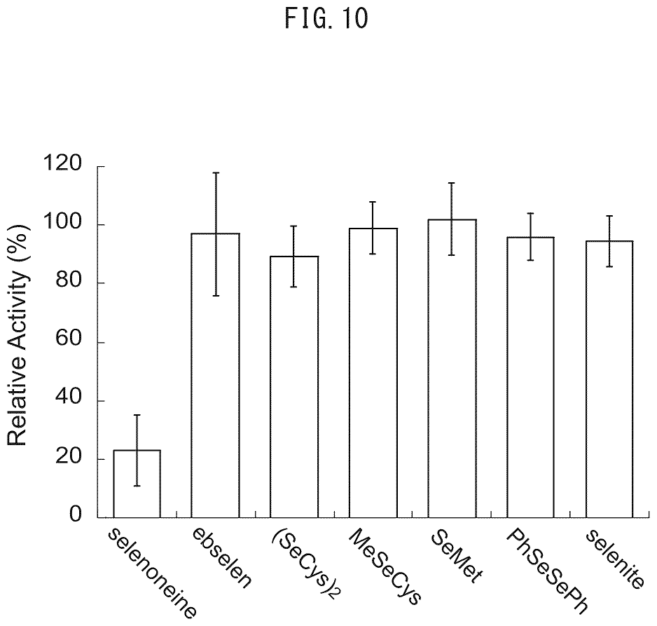

After dissolving 2 μg/ml M^{pro} and 1 μM test compound in 50 mM Tris-HCl (pH 7.4) kept at 37° C., preincubation was carried out for 5 minutes. Thereafter, 10 μM Ac-Abu-Tle-Leu-Gln-MCA was added to the reaction solution, and the fluorescence intensity resulting from cleavage of Ac-Abu-Tle-Leu-Gln-MCA was measured using a fluorometer, to measure the inhibitory activity (FIG. 10). As the test compound, selenoneine, ebselen, selenocystine ((SeCys)₂), methylselenocysteine (MeSeCys), selenomethionine (SeMet), diphenyl diselenide (PhSeSePh), or sodium selenite was used. These serine-containing compounds with the exception of sodium selenite are compounds that were predicted to have M^{pro} inhibitory activity in an in-silico test (NPL 4). When the fluorescence intensity observed in the case without addition of a test compound was taken as 100, the fluorescence intensity measured 1 minute after the addition of the test compound was about 20% in the case of selenoneine. On the other hand, in all of the cases where ebselen, selenocystine ((SeCys)₂), methylselenocysteine (MeSeCys), selenomethionine (SeMet), diphenyl diselenide (PhSeSePh), or sodium selenite (selenite) was added, the fluorescence intensity was more than 90%. Thus, selenoneine was shown to have a remarkably higher M^{pro} inhibitory activity than the other selenium-containing compounds.

### Example 6: Growth Inhibitory Activity against SARS-COV2

Inhibition of SARS-COV2 infection to host cells, by ebselen or selenoneine can be measured by, for example, the plaque assay method. Host cells are subjected to monolayer culture to confluence in an incubator at 37° C. and 5% CO₂. The medium is then removed, and the cell surface is washed with PBS (−), followed by adding a virus solution and 0 to 100 μM protease inhibitor solution to the cells and performing incubation for 30 minutes. Thirty minutes later, the sample solution is removed, and an agar medium is layered on the cells. After solidification of the agar, the plate is placed in an inverted position, and incubated for 2 days in an incubator at 37° C. and 5% CO₂. Thereafter, the monolayer medium is removed, and the plate is dried, followed by performing staining with a crystal violet staining solution for 5 minutes, washing the plate with purified water, and then air-drying the plate. Finally, the number of plaques is counted and compared to that of a control group, to calculate the rate of infection inhibition by the inhibiting agent for protease against the virus.

1. A method for inhibiting protease of coronavirus, comprising contacting selenoneine, a tautomer or dimer thereof, or a pharmaceutically acceptable salt thereof with coronavirus.

2. The method according to claim 1, wherein the protease is main protease or papain-like protease.

3. The method according to claim 1, wherein the coronavirus is SARS-COV2.

4. A method for treatment or prevention of coronavirus infection in a subject, comprising administering selenoneine, a tautomer or dimer thereof, or a pharmaceutically acceptable salt thereof to the subject.

5. The method according to claim 4, wherein the coronavirus infection is COVID-19.

6. A method for treatment or prevention of coronavirus infection in a subject through inhibition of protease of coronavirus, comprising administering selenoneine, a tautomer or dimer thereof, or a pharmaceutically acceptable salt thereof to the subject.

7. A method of screening a therapeutic or prophylactic agent for coronavirus infection, using an inhibitory activity against papain as an index.

8. The method according to claim 7, wherein the coronavirus infection is COVID-19.

9. The method according to claim 7, wherein the therapeutic agent or prophylactic agent inhibits main protease of coronavirus.

10. The method according to claim 6, wherein the protease is main protease or papain-like protease.

11. The method according to claim 6, wherein the coronavirus is SARS-COV2.


SEQUENCE LISTING (5 sequences)

SEQ ID NO: 1 - protein, 306 aa, SARS-CoV-2 [sequence omitted]

SEQ ID NO: 2 - protein, 345 aa, Carica papaya
MAMIPSISKLLFVAICLFVYMGLSFGDFSIVGYSQNDLTSTERLIQLFESWMLKHNKIYKNIDEKIYRFEIFKDNLKYIDETNKKNNSYWLGLNVFADMSNDEFKEKYTGSIAGNYTTTELSYEEVLNDGDVNIPEYVDWRQKGAVTPVKNQGSCGSCWAFSAVVTIEGIIKIRTGNLNEYSEQELLDCDRRSYGCNGGYPWSALQLVAQYGIHYRNTYPYEGVQRYCRSREKGPYAAKTDGVRQVQPYNEGALLYSIANQPVSVVLEAAGKDFQLYRGGIFVGPCGNKVDHAVAAVGYGPNYILIKNSWGTGWGENGYIRIKRGTGNSYGVCGLYTSSFYPVKN

SEQ ID NO: 3 - protein, 317 aa, SARS-CoV-2
EVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKAA

SEQ ID NO: 4 - DNA, 918 bp, SARS-CoV-2
agtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaa

SEQ ID NO: 5 - DNA, 954 bp, SARS-CoV-2
gaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttact